(19) United States
(12) Patent Application Publication
(10) Pub. No.: US 2021/0267977 A1
(43) Pub. Date: Sep. 2, 2021

(54) CONTROLLING EFFECTS AFTER 5HT2A AGONISTS ADMINISTRATION

(71) Applicant: Universitätsspital Basel, Basel (CH)

(72) Inventor: Matthias Emanuel LIECHTI, Oberwil (CH)

(21) Appl. No.: 17/156,233

(22) Filed: Jan. 22, 2021

Related U.S. Application Data
(60) Provisional application No. 62/982,877, filed on Feb. 28, 2020.

(51) Int. Cl. A61K 31/517; A61K 31/48; A61P 25/26

(52) U.S. Cl. CPC A61K 31/517; A61P 25/26; A61K 31/48

(57) ABSTRACT

A composition for treating an individual while reducing acute effects, including effective amounts of a psychedelic drug and a duration shortening agent. A method of treating an individual with a psychedelic drug and reducing its acute duration of action, by administering a psychedelic drug to the individual, administering a duration shortening agent to the individual, and shortening and/or reducing the acute effects of the psychedelic drug. A method of stopping the acute duration of action of a psychedelic drug in an individual, by administering a duration shortening agent to the individual after the individual has taken a psychedelic drug and stopping the acute effects of the psychedelic drug.

### GRANT INFORMATION

Research in this application was supported in part by a grant from the Swiss National Science Foundation (Grant No. 320036_185111).

### BACKGROUND OF THE INVENTION

### 1. Technical Field

The present invention relates to compositions and methods for using 5HT2A antagonists in medical treatments. More specifically, the present invention relates to methods and means for shortening acute effects of 5HT2A agonists.

### 2. Background Art

Lysergic acid diethylamide (LSD) can be used to assist psychotherapy for many indications including anxiety, depression, addiction, personality disorder and others and can also be used to treat other disorders such as cluster headache and migraine and others (Hintzen & Passie, 2010; Liechti, 2017; Nichols, 2016; Passie et al., 2008). LSD targets the 5HT2A receptor, which is a serotonin receptor. Effects of LSD can include altered thoughts, feelings, awareness of surroundings, dilated pupils, increased blood pressure, and increased body temperature.

The duration of action of LSD is long. Doses commonly used in LSD-assisted treatment/psychotherapy are 100-200 μg. A dose of 100 μg produced subjective effects in humans lasting (mean±SD) 8.5±2.0 hours (range: 5.3-12.8 hour) in one representative study (Holze et al., 2019). In other studies, LSD effects similarly lasted 8.2±2.1 hours (range: 5-14 hours) after administration of a 100 μg dose and 11.6±1.7 hours (range: 7-19.5 hours) after administration of a 200 μg dose (Dolder et al., 2017).

The dose-dependent and long duration of action of LSD can be a problem in certain treatment settings. Patients need to be supervised closely and this consumes resources (time, personnel). Additionally, some patients prefer shorter treatments. Further, some patients may also not tolerate the treatment well, in which case a shorter treatment would be needed or a shortening of a treatment that has already started would be needed.

In the past, the problem of the long duration of action of LSD was addressed and partly solved by replacing LSD with shorter-acting substances to assist psychotherapy. In the majority of cases, LSD was replaced by psilocybin which acts for approximately 4-6 hours (Griffiths et al., 2016; Passie et al., 2008) and therefore has a duration of action that is approximately half as long as that of LSD at equivalently psychoactive doses. In part, as a result of the long duration of action of LSD, psilocybin has been used in most of the recent clinical research trials evaluating the efficacy of psychedelics to assess psychotherapy (Carhart-Harris et al., 2017; Carhart-Harris et al., 2016; Griffiths et al., 2016; Grob et al., 2011; Ross et al., 2016). However, a few studies have also used LSD despite its long duration of action (Gasser et al., 2014; Gasser et al., 2015). Additionally, some physicians and patients want to use LSD rather than psilocybin. For example, there is considerably more data on the use and associated safety of LSD than psilocybin. In fact, LSD was mostly used in the 1940's-1970's while psilocybin was only studied more recently mainly after 2000.

U.S. Patent Application Publication No. 20200397752 to Perez Castillo, et al. discloses a combination product for the treatment and/or prevention of psychiatric and/or neurological disorders. The combination product comprises (i) a compound which promotes neurogenesis and has hallucinogenic and/or psychedelic side effects, and (ii) a 5-HT2A receptor antagonist which alleviates and/or removes the hallucinogenic and/or psychedelic side effects caused by the first compound. The 5-HT2A receptor antagonist can be ketanserin. The studies were performed using the hallucinogen dimethyltryptamine (DMT) in animals and to produce neurogenesis and a combination of DMT and ketanserin was used. The invention claims the use of a combination product with exclusively a tryptamine and any 5-HT2A antagonist including ketanserin for use as a medicament. The aim of this treatment is to induce neurogenesis without psychotropic effects. In contrast, the present studies in humans and the present invention have the goal of inducing a full psychedelic experience in humans using any psychedelic and using ketanserin or any 5-HT2A receptor antagonist to shorten or abort the psychedelic experience.

There remains a need for a safe and effective method of using LSD as well as other 5HT2A agonists.

### SUMMARY OF THE INVENTION

The present invention provides for a composition for treating an individual while reducing acute effects, including effective amounts of a psychedelic drug and a duration shortening agent.

The present invention provides for a method of treating an individual with a psychedelic drug and reducing its acute duration of action, by administering a psychedelic drug to the individual, administering a duration shortening agent to the individual, and shortening and/or reducing the acute effects of the psychedelic drug.

The present invention also provides for a method of stopping the acute duration of action of a psychedelic drug in an individual, by administering a duration shortening agent to the individual after the individual has taken a psychedelic drug and stopping the acute effects of the psychedelic drug.

### DESCRIPTION OF THE DRAWINGS

Other advantages of the present invention are readily appreciated as the same becomes better understood by reference to the following detailed description when considered in connection with the accompanying drawings wherein:

FIG. 1 shows a representation of LSD;

FIG. 2 shows a representation of ketanserin;

FIG. 3 is a graph of an effect-time curve of LSD alone;

FIG. 4 is a graph of an effect-time curve of LSD with ketanserin administered after LSD;

FIG. 5A is a graph of any drug effect versus time, FIG. 5B is a graph of good drug effect versus time, FIG. 5C is a graph of bad drug effect versus time, FIG. 5D is a graph of drug liking versus time, FIG. 5E is a graph of stimulated versus time, FIG. 5F is a graph of fear versus time, FIG. 5G is a graph of ego dissolution versus time, FIG. 5H is a graph of sense of time versus time, and FIG. 5I is a graph of concentration versus time;

FIG. 6A is a graph of well-being versus time, FIG. 6B is a graph of anxiety versus time, FIG. 6C is a graph of inactivity versus time, FIG. 6D is a graph of extraversion versus time, FIG. 6E is a graph of introversion versus time, and FIG. 6F is a graph of emotional excitation over time;

FIG. 7 is a table of data of the comparison of the acute effects of LSD alone, LSD+ketanserin, and placebo;

FIG. 8A is a graph of oceanic boundlessness, FIG. 8B is a graph of anxious ego dissolution, and FIG. 8C is a graph of visionary restructualisation;

FIG. 9 is a graph of % scale maximum;

FIG. 10A is a graph of systolic blood pressure versus time, FIG. 10B is a graph of diastolic blood pressure versus time, FIG. 10C is a graph of heart rate versus time, and FIG. 10D is a graph of body temperature versus time;

FIG. 11 is a table of acute adverse drug effects;

FIG. 12 is a table of pharmacokinetic parameters for LSD based on compartmental modeling;

FIG. 13 is a table of blinding data;

FIGS. 14A-141 are graphs of subjective effects of LSD with ketanserin or placebo administered 1 hour after LSD, FIG. 14A shows “any drug effect”, FIG. 14B shows “good drug effect”, FIG. 14C shows “visual perception alterations”, FIG. 14D shows “sounds influenced what I saw (synesthesia)”, FIG. 14E shows “alterations in the sense of time”, FIG. 14F shows “ego-dissolution”, FIG. 14G shows feelings of stimulation, FIG. 14H shows feelings of tiredness, and FIG. 14I shows nausea;

FIGS. 15A and 15B are graphs showing LSD-induced alterations of the mind after additional administration of ketanserin or placebo 1 hour after LSD; and

FIGS. 16A-16D are graphs of cardiovascular effects of LSD after additional administration of ketanserin or placebo 1 hour after administration of LSD, FIG. 16A shows systolic blood pressure, FIG. 16B shows diastolic blood pressure, FIG. 16C shows heart rate, and FIG. 16D shows body temperature.

### DETAILED DESCRIPTION OF THE INVENTION

The present invention provides generally for short-acting psychedelic treatments for medical conditions. More specifically, the present invention provides for a composition for treating an individual while reducing acute effects, including effective amounts of a psychedelic drug and a duration shortening agent, most preferably ketanserin. In general, the ketanserin reduces the acute effects of the psychedelic drug.

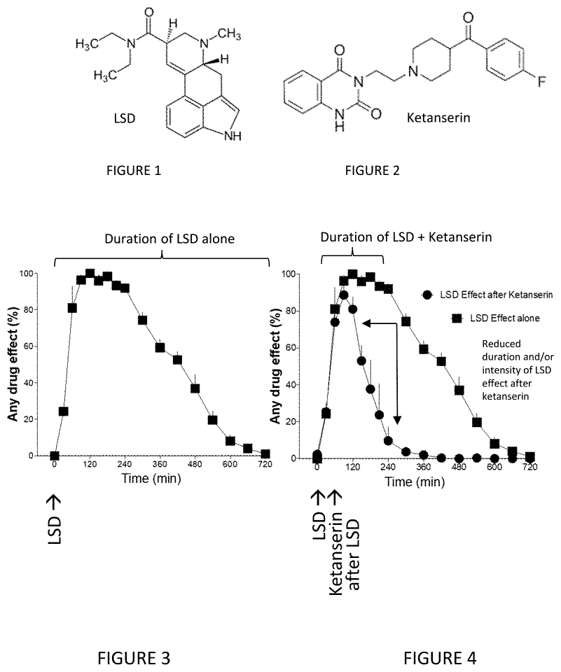

The psychedelic drug can be, but is not limited to, 5HT2A agonists such as LSD, psilocybin, mescaline, dimethyltryptamine (DMT), 2,5-dimethoxy-4-iodoamphetamine (DOI), 2,5-dimethoxy-4-bromoamphetamie (DOB), salts thereof, analogs thereof, or homologues thereof. Preferably, the dose of the psychedelic is one that provides an effect for at least a few hours (such as at least 2 hours) or has a meaningful effect. A dose of 0.01-1 mg (10-1000 μg can be used of LSD but antagonisms is most meaningful if higher doses of LSD (>0.05 mg) or a psychedelic are used. FIG. 1 shows a representation of LSD. Psilocybin can be dosed at 10-50 mg, mescaline can be dosed at 100-800 mg, DMT can be dosed at 20-100 mg, DOI can be dosed at 0.1-5 mg, and DOB can be dosed at 0.1-5 mg. With dose ranges including also very high doses not commonly used clinically in these examples.

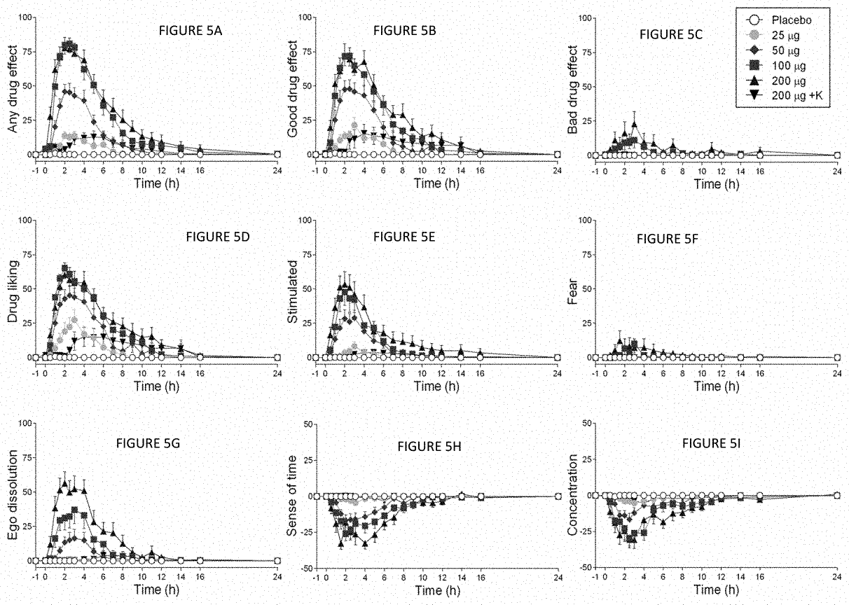

The duration shortening agent can be any suitable agent that is able to reduce the acute effects of the psychedelic drug and is preferably a 5HT2A receptor antagonist such as ketanserin, salts thereof, analogs thereof, and homologs thereof. Ketanserin is an antihypertensive agent and is a high-affinity antagonist of 5HT2A. As described above, the 5HT2A receptor is a serotonin receptor and G protein-coupled receptor that is a target of serotonergic psychedelic drugs like LSD. Ketanserin has been used as a radioligand for serotonin 5HT2 receptors. Ketanserin is also a high affinity antagonist for the H1 receptor. The antihypertensive response of ketanserin is due to blockade of the alpha1-adrenoceptor as well as blockade of 5HT2A. A dose of 5-100 mg can be used. FIG. 2 shows a representation of ketanserin. The duration shortening agent can also be an effect blocking agent.

Most preferably, the compounds are provided separately and administered orally, however, they can also be provided in the same dosage unit and have the same or different release profiles. For example, the dosage unit can be designed to release the psychedelic drug first and subsequently at a later time release the ketanserin.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intramuscular, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of treating an individual with a psychedelic drug and reducing its acute duration of action, by administering a psychedelic drug to the individual, administering a duration shortening agent such as ketanserin to the individual, and shortening and/or reducing the acute effects of the psychedelic drug. The psychedelic drug can be administered in amounts of 0.01-1 mg for LSD and ketanserin can be administered in amounts of 5-100 mg. In Example 3, LSD was administered at 100 μg and ketanserin was administered at 40 mg. Ketanserin can be administered 1 minute to 24 hours after the administration of the psychedelic drug. In Example 3, ketanserin was administered 1 hour after LSD. Any of the psychedelic drugs described above can be used in this method. The administration of the duration shortening agent can be at the same time as or at a later time than administration of the psychedelic drug, depending on the formulation. The administration steps can be accomplished by separate oral administration, or as described above, with a single oral dosage unit with release of the psychedelic drug first and subsequent release of the ketanserin.

The method can also include the step of reducing the time of subjective effects including any drug effect, bad drug effect, anxiety, ego-dissolution, or any other subjective response measure or any other related autonomic response measure (blood pressure, heart rate, or/and pupil size) by 10-100% compared with a treatment consisting of the same amount of the psychedelic drug alone. In Example 3, ketanserin reduced effects of LSD one hour after administration and blocked LSD effects within two hours to return the individuals to approximately a normal state. Also, there is no recurrence of the psychedelic drug effects after ketanserin is administered. In other words, the ketanserin remain efficacious in the body of the individual.

The method can be used to reduce time and/or degree of cognitive impairment due to the psychedelic drug, reduce time of treatment session supervision by medical personnel, reduce intensity and/or duration of anxiety or any other acute adverse effects in response to the psychedelic drug, reduce expected acute adverse effects intensity and/or duration due to inadvertent administration of a high dose of the psychedelic drug, reduce expected acute adverse effects intensity and/or duration due to intentional intake of the psychedelic drug (overdose), and reduce expected acute adverse effects duration and/or intensity due to intentional intake of the psychedelic drug in doses considered too high or producing too strong effects after administration.

The present invention also provides for a method of stopping the acute duration of action of a psychedelic drug in an individual, by administering a duration shortening agent such as ketanserin to the individual after the individual has taken a psychedelic drug, and stopping the acute effects of the psychedelic drug. As also described below, this method can be useful in stopping effects of psychedelic drugs that are having an adverse effect on an individual or in the case of an overdose. The duration shortening agent is efficacious in stopping acute effects of the psychedelic when administered after the psychedelic.

The invention allows the psychedelic drug experience to be modified (attenuated) with the goal of reducing the acute subjective psychedelic drug effect duration with the goal of 1) reducing time of supervision and 2) avoiding prolonged negative acute treatment effects. For example, the invention targets a reduction of the time of action by 50% to 4-6 hours compared to the classic treatment with LSD alone and reaching a similar duration of action as with psilocybin. The use of a pharmacological antagonist such as ketanserin (40 mg orally) 1 hour prior to the oral administration of LSD at a moderate dose (70-100 μg) has been shown to prevent the LSD experience almost completely (Preller et al., 2017). Administration of ketanserin (40 mg orally) 1 hour prior to a high dose of LSD of 200 μg similarly prevented the LSD experience (Liechti). The present invention uses ketanserin after administration of the psychedelic drug to shorten the psychedelic drug experience. LSD primarily binds to and activates the serotonin 5HT2A receptor (Rickli et al., 2016) and this receptor interaction is prevented by the 5HT2A receptor antagonist ketanserin which potently binds to this receptor. Researchers have shown strong and unique binding of LSD to the receptor and stated that this process is underlying the long duration of action of LSD in humans (Wacker et al., 2017). Others have shown that LSD acts only as long as it is present in the body and that therefore no special mechanisms at the receptor would be needed to explain its duration of action in humans. Rather the duration is explained well by its pharmacokinetic characteristics (Holze et al., 2019). Importantly, there seems to be significant controversy about whether simple binding of LSD to its target receptor is sufficient to explain its duration of action and therefore it is not obvious that administering a receptor antagonist such as ketanserin would attenuate and shorten the action of LSD in humans. Thus, it is not obvious that the LSD experience can be blocked with a treatment performed after administration based on the known information that ketanserin can prevent an LSD response when ketanserin was administered 1 hour before the LSD.

There are several advantages to the present invention. The action of psychedelic drugs such as LSD that is usually long (8-12 hours) can be made shorter (2-6 hours), allowing shorter and more cost-effective treatment session. In these cases, ketanserin can be administered 1-2 hours after the psychedelic drug to shorten the duration of action by 2-6 hours as to be shown by supporting studies. The present invention can also attenuate or even stop the psychedelic drug experience using ketanserin to treat patients who a) do not respond well to psychedelic drugs (horror trip), b) consider the experience as too strong, or c) were overdosed. In all these cases, ketanserin can be given immediately after the need to attenuate/antagonize the psychedelic drug effects becomes evident. In extreme cases, ketanserin can be given immediately after the psychedelic drug.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

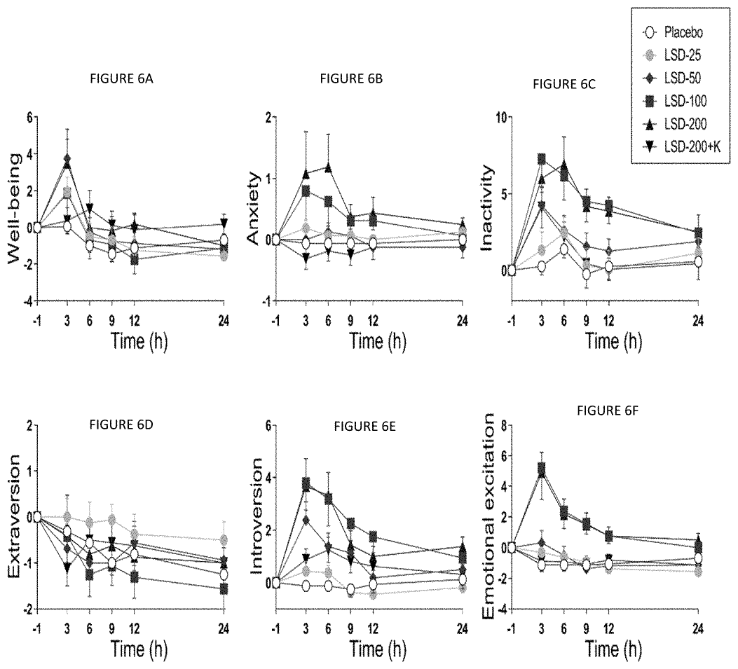

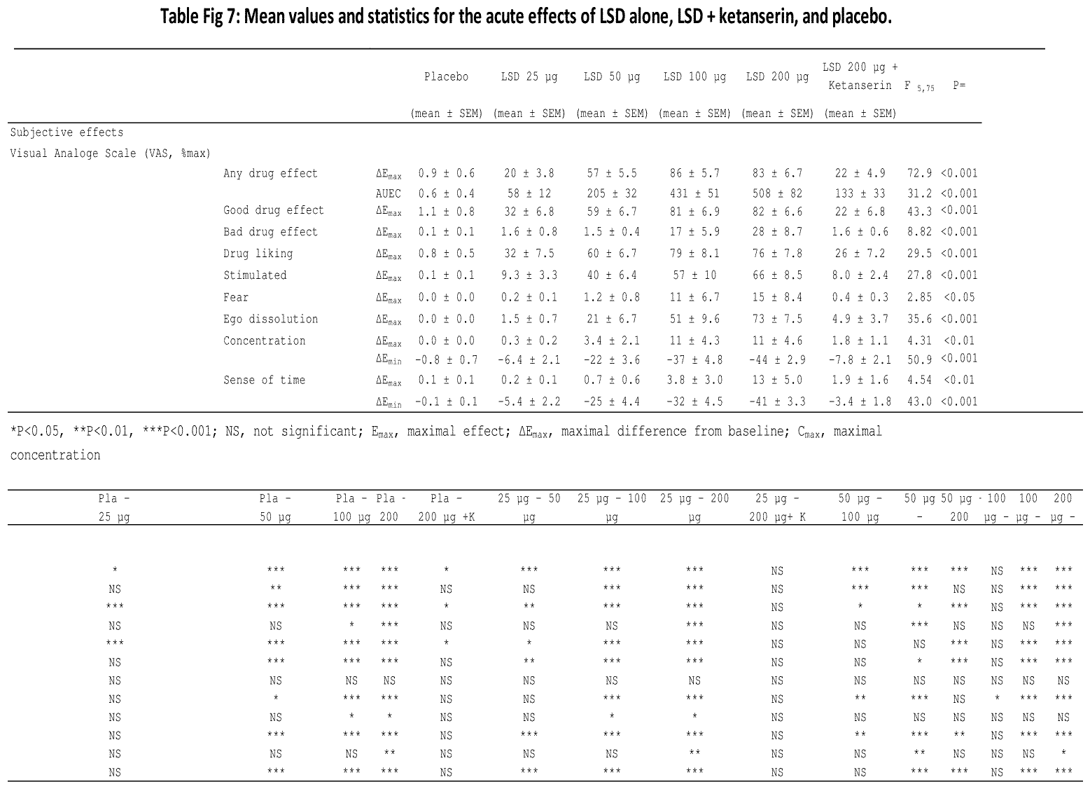

FIG. 3 shows the effect-time curve of LSD alone. FIG. 4 shows the effect-time curve of LSD with ketanserin administered after LSD as per the present invention. As can be seen in FIG. 4, there is a reduced duration and/or intensity of the LSD effect after the administration of ketanserin. The curves are illustrations of the invention and are derived from pharmacological information on LSD and ketanserin including the studies described under EXAMPLE 2 and EXAMPLE 3 conducted to generate the present invention. Details on the acute effects of LSD and ketanserin are shown below.

### Example 2

A clinical study administered ketanserin prior to a high dose of LSD to document that high LSD dose effects can be antagonized with ketanserin. While the present invention administers ketanserin after LSD, this Example shows that the two drugs work together to reduce acute effects. The data used in EXAMPLE 2 and describing parts of the present invention has been published in (Holze et al., 2020).

The key results are:

Ketanserin markedly and significantly (most P<0.001) reduced the subjective response to high-dose LSD approximately to the level of the 25 μg LSD dose. Ketanserin significantly prevented the LSD-induce heart rate response. Ketanserin significantly prevented the acute adverse effects of 200 μg LSD. Ketanserin only minimally altered the PK of 200 μg LSD. Ketanserin and LSD together were identified correctly or mistaken as a low dose of LSD but never mistaken for a high dose of LSD.

Materials and Methods

Study design: The study used a double-blind, placebo-controlled, cross-over design with six experimental test sessions to investigate the responses to 1) placebo 2) 25 μg, 3) 50 μg, 4) 100 μg, 5) 200 μg LSD and 6) 200 μg LSD after ketanserin (40 mg). The washout periods between sessions were at least 10 days. The study was registered at ClinicalTrials.gov (NCT03321136).

Participants: Sixteen healthy subjects (eight men and eight women; mean age±SD: 29±6.4 years; range: 25-52 years) were recruited. Participants who were younger than 25 years old were excluded from participating in the study. Additional exclusion criteria were age >65 years, pregnancy (urine pregnancy test at screening and before each test session), personal or family (first-degree relative) history of major psychiatric disorders (assessed by the Semi-structured Clinical Interview for Diagnostic and Statistical Manual of Mental Disorders, 4th edition, Axis I disorders by a trained psychiatrist), the use of medications that may interfere with the study medications (e.g. antidepressants, antipsychotics, sedatives), chronic or acute physical illness (abnormal physical exam, electrocardiogram, or hematological and chemical blood analyses), tobacco smoking (>10 cigarettes/day), lifetime prevalence of illicit drug use >10 times (except for Δ⁹-tetrahydrocannabinol), illicit drug use within the last 2 months, and illicit drug use during the study (determined by urine drug tests).

Study drugs: LSD (D-lysergic acid diethylamide base, high-performance liquid chromatography purity >99%; Lipomed AG, Arlesheim, Switzerland) was administered as oral solution in units containing 100 (Holze et al., 2019) or 25 μg LSD in 1 mL of 96% ethanol. Thus, subjects ingested 2 mL of LSD solution and/or placebo (96% ethanol) per session: 1) placebo/placebo, 2) 25 μg LSD/placebo, 3) 25 μg LSD/25 μg LSD, 4) 100 μg LSD/placebo, 5) 100 μg LSD/100 μg LSD, 6. 100 μg LSD/100 μg LSD). Ketanserin was obtained as the marketed drug (KETENSIN® (Janssen)) and encapsulated with opaque capsules to ensure blinding. Placebo consisted of identical opaque capsules filled with mannitol. Thus, blinding to treatment was guaranteed by using a double-dummy method, with identical capsules and vials that were filled with mannitol and ethanol, respectively, as placebo. At the end of each session and at the end of the study, the participants were asked to retrospectively guess their treatment assignment.

Study procedures: The study included a screening visit, six 25 hour test sessions, and an end-of-study visit. Ketanserin (40 mg) or placebo was administered at 8:00 AM. LSD or placebo was administered at 9:00 AM. The outcome measures were repeatedly assessed for 24 hours.

Subjective drug effects: Subjective effects were assessed repeatedly using visual analog scales (VASs) 1 hour before and 0, 0.5, 1, 1.5, 2, 2.5, 3, 4, 5, 6, 7, 8, 9, 10, 11, 12, 14, 16, and 24 hours after LSD administration. The VASs were presented as 100-mm horizontal lines (0-100%), marked from “not at all” on the left to “extremely” on the right. The VASs for “concentration”, and “perception of time” were bidirectional (±50%). Marked from “not at all” on the left (−50), to “normal” in the middle (0), to “extremely” on the right (+50) for concentration and “slowed” (−50) and “racing” (+50) for “perception of time”. The 5D-ASC scale (Dittrich, 1998; Studerus et al., 2010) was administered 24 hours after LSD administration to retrospectively rate alterations in waking consciousness induced by the drugs. Mystical experiences were assessed using the German version (Liechti et al., 2017) of the 100-item States of Consciousness Questionnaire (SOCQ) (Griffiths et al., 2006) that includes the 43-item and newer 30-item MEQ (MEQ43 (Griffiths et al., 2006) and MEQ30 (Barrett et al., 2015)). The 60-item Adjective Mood Rating Scale (AMRS) (Janke & Debus, 1978) was administered 1 hour before and 3, 6, 9, 12, and 24 hours after drug administration.

Autonomic, adverse, and endocrine effects: Blood pressure, heart rate, and tympanic body temperature were repeatedly measured 1 hour before and 0, 0.5, 1, 1.5, 2.5, 3, 4, 5, 6, 7, 8, 9, 10, 11, 12, 14, 16, and 24 hours after drug administration as previously described in detail (Hysek et al., 2010). Adverse effects were systematically assessed 1 hour before and 12 and 24 hours after drug administration using the 66-item List of Complaints (Zerssen, 1976).

Plasma drug concentrations: Blood was collected into lithium heparin tubes 1 hour before and 0, 0.5, 1, 2, 3, 4, 6, 8, 10, 12, 14, 16, and 24 hours after LSD administration. The blood samples were immediately centrifuged, and the plasma was subsequently stored at −80° C. until analysis. Plasma concentrations of LSD and O-H-LSD were determined using a validated ultra-high-performance liquid chromatography tandem mass spectrometry method as described previously in detail (Holze et al., 2019).

Pharmacokinetic analyses and pharmacokinetic-pharmacodynamic modeling: Pharmacokinetic parameters were estimated using a one-compartment model with first-order input, first-order elimination, and no lag time in Phoenix WinNonlin 6.4 (Certara, Princeton, N.J., USA) as described previously in detail (Holze et al., 2019).

Data analysis: Peak (Eₘₐₓ and/or Eₘᵢₙ) or peak change from baseline (ΔEₘₐₓ) values were determined for repeated measures. The values were then analyzed using repeated-measures analysis of variance (ANOVA), with drug as within-subjects factor, followed by Tukey post hoc comparisons using Statistica 12 software (StatSoft, Tulsa, Okla., USA). The criterion for significance was p<0.05.

Results

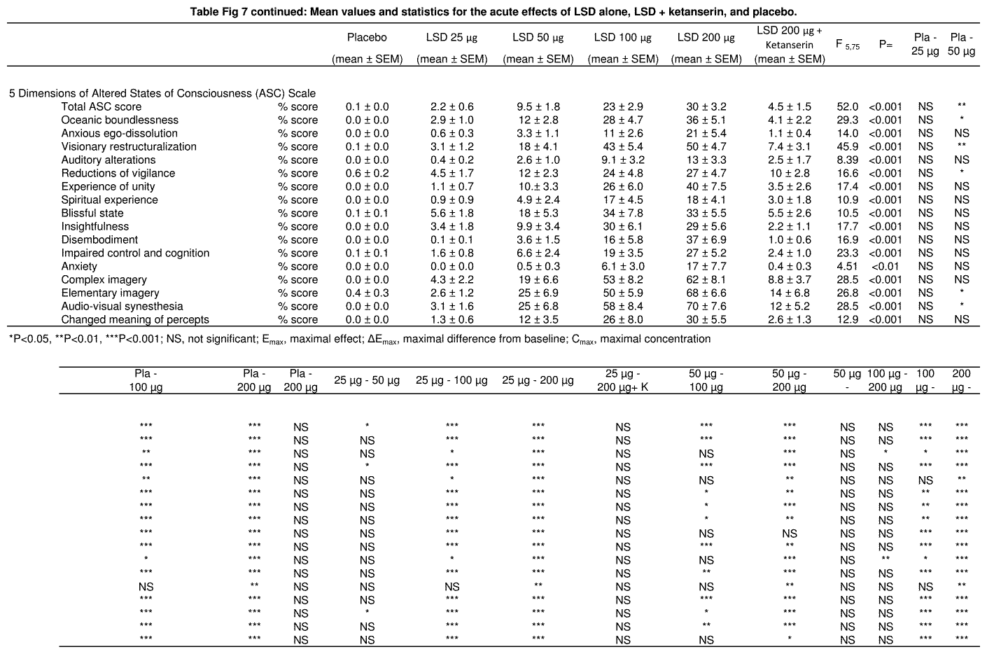

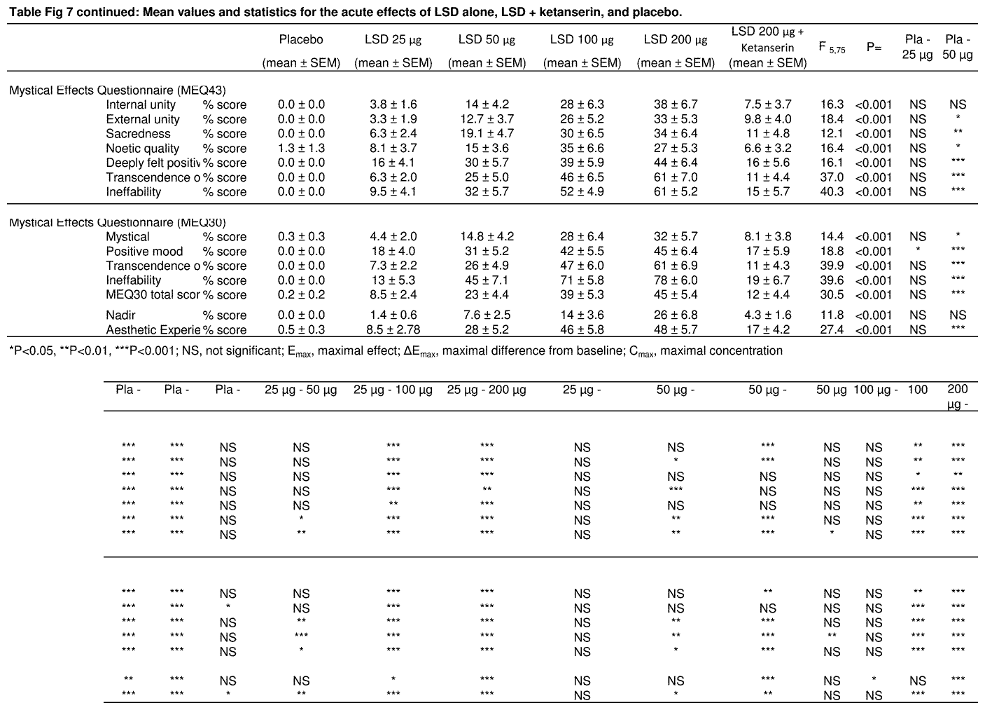

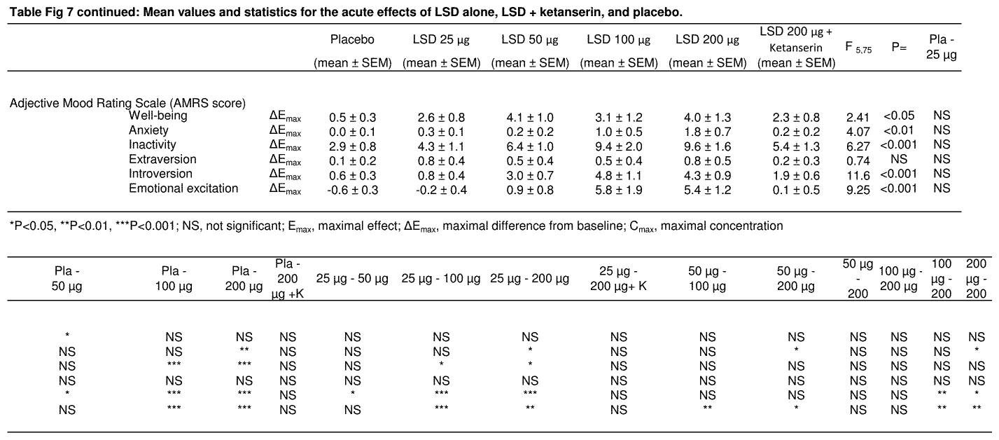

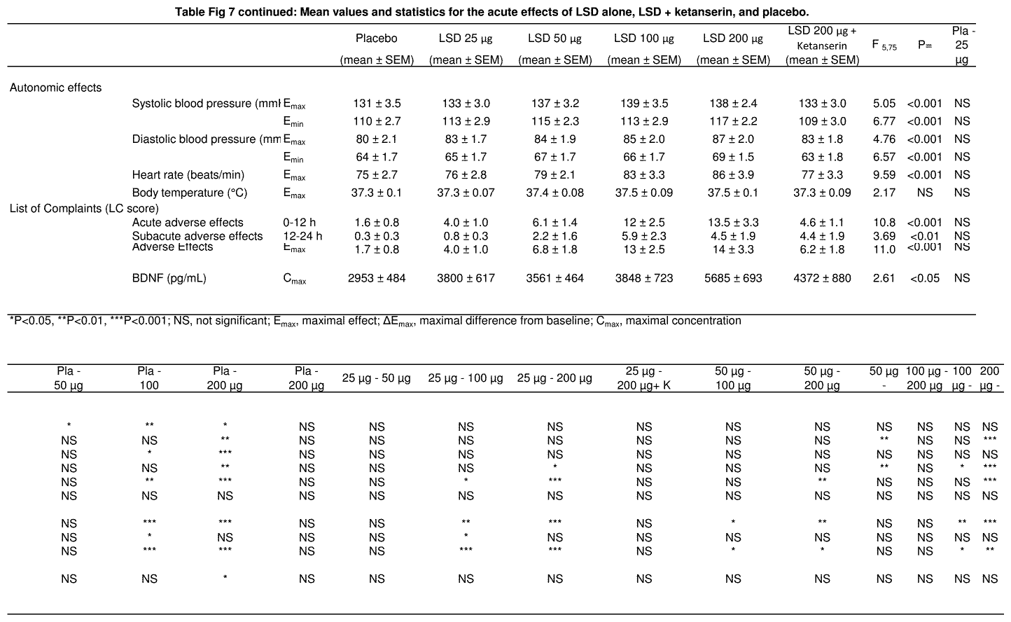

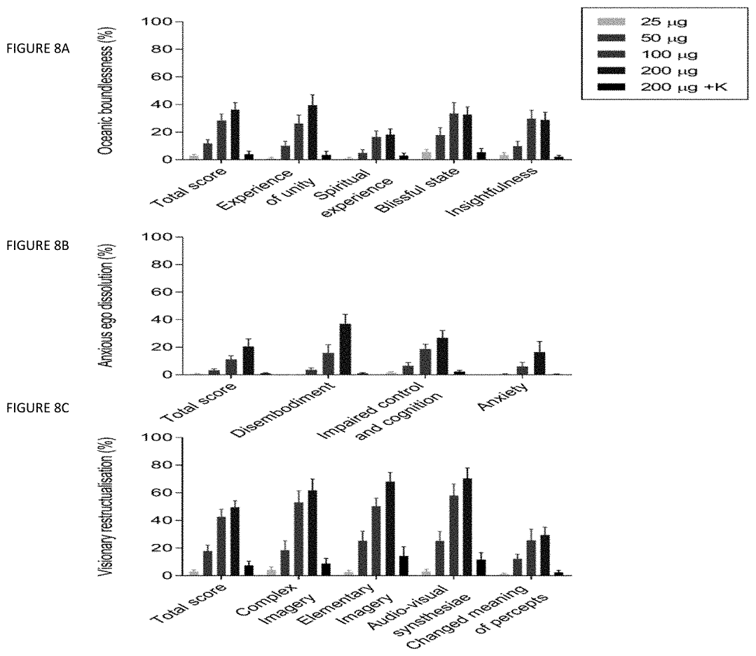

Subjective drug effects: Subjective effects over time on the VAS and AMRS are shown in FIGS. 5A-5I and FIGS. 6A-6F, respectively. Ketanserin or placebo was administered at t=−1 hour and LSD or placebo was administered at t=0 hour. The data are expressed as the mean±SEM % maximal values in 16 subjects. FIGS. 6A-6F show that ketanserin blocked most of the LSD responds on the AMRS. Specifically, ketanserin significantly prevented anxiety, introversion and emotional excitation induced by 200 μg LSD. The corresponding peak responses and statistics are presented in FIG. 7. Alterations of mind and mystical-type effects are shown in FIGS. 8A-8C and FIG. 9, respectively, and statistics in FIG. 7. FIGS. 8A-8C show that ketanserin significantly and markedly reduced the subjective effects on the 5 Dimensions of Altered States of Consciousness (5D-ASC) Scale to 200 μg LSD to the level of 25 μg LSD. The data are expressed as the mean±SEM % scale maximum values in 16 subjects. Ratings for placebo are not expressed here, because ratings are too low for visualization. FIG. 9 shows that ketanserin significantly and markedly reduced the subjective effects on the Mystical Effects Questionnaire (MEQ) and the Subscales ‘Nadir’ and ‘Aesthetic experience’ derived from the SOCQ to 200 μg LSD to the level of 25 μg LSD. The data are expressed as the mean±SEM % scale maximum values in 16 subjects. Ratings for placebo are not expressed here, because ratings are too low for visualization. Overall, ketanserin markedly and significantly (most P<0.001) reduced the subjective response to high-dose LSD approximately to the level of the 25 μg LSD dose.

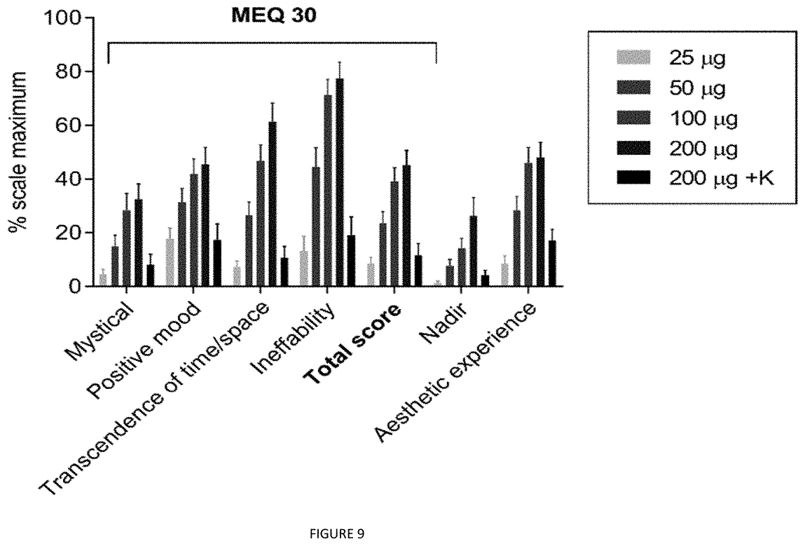

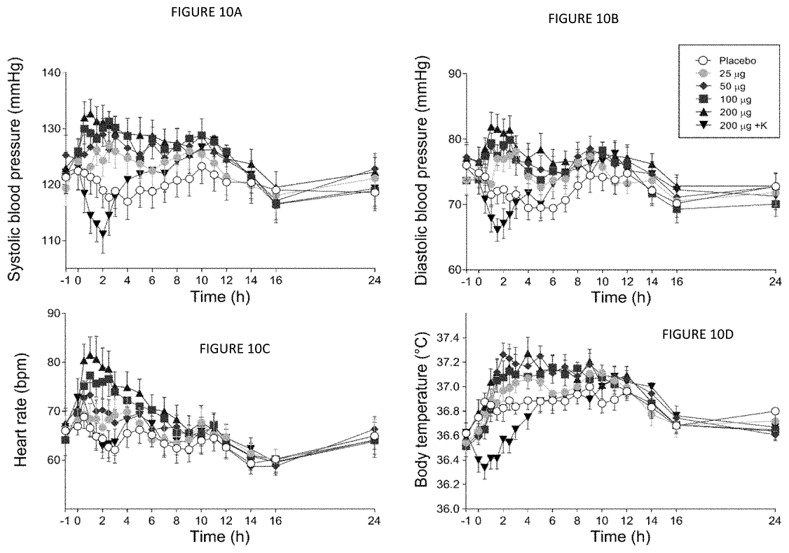

Cardiovascular, autonomic, adverse, and endocrine effects: Autonomic effects over time and the respective peak effects are shown in FIGS. 10A-10D and FIG. 7, respectively. FIGS. 10A-10D show that LSD+Ketanserin shows an transient decrease for systolic blood pressure, diastolic blood pressure, heart rate, and body temperature with a later increase up to the level of LSD for the systolic and diastolic blood pressure and a normalization for heart rate and body temperature. The data are expressed as the mean±SEM in 16 subjects. Ketanserin significantly prevented the LSD-induce heart rate response. Ketanserin transiently reduced the LSD response on blood pressure only up to 6 hours. Ketanserin significantly prevented the acute adverse effects of 200 μg LSD. Frequently reported adverse effects are presented in FIG. 11.

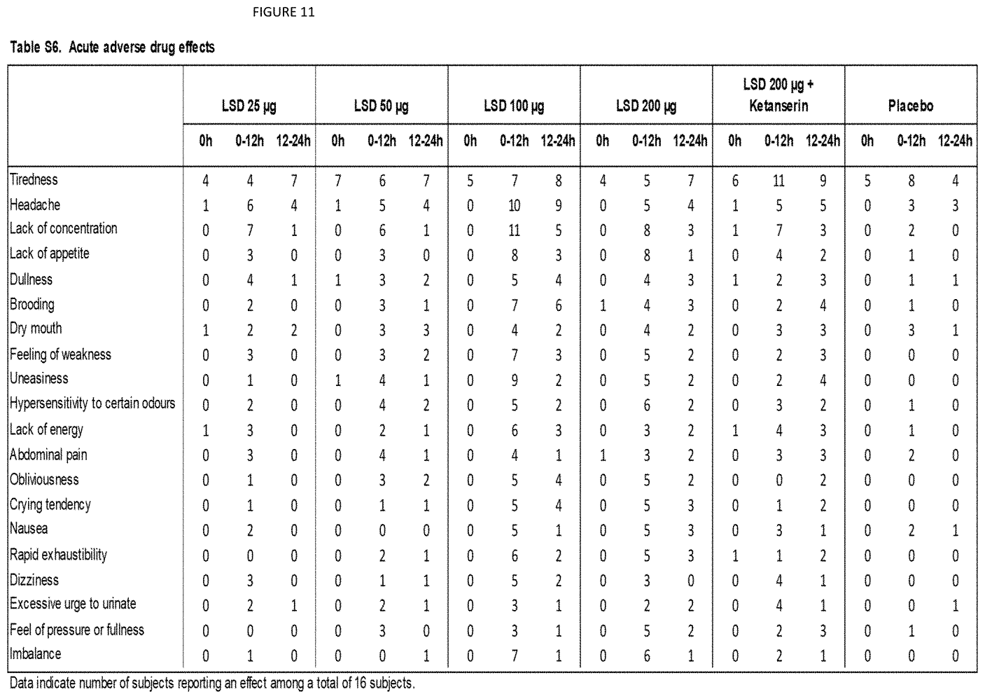

Pharmacokinetics: FIG. 12 shows the pharmacokinetic parameters of LSD. Ketanserin only minimally altered the PK of 200 μg LSD.

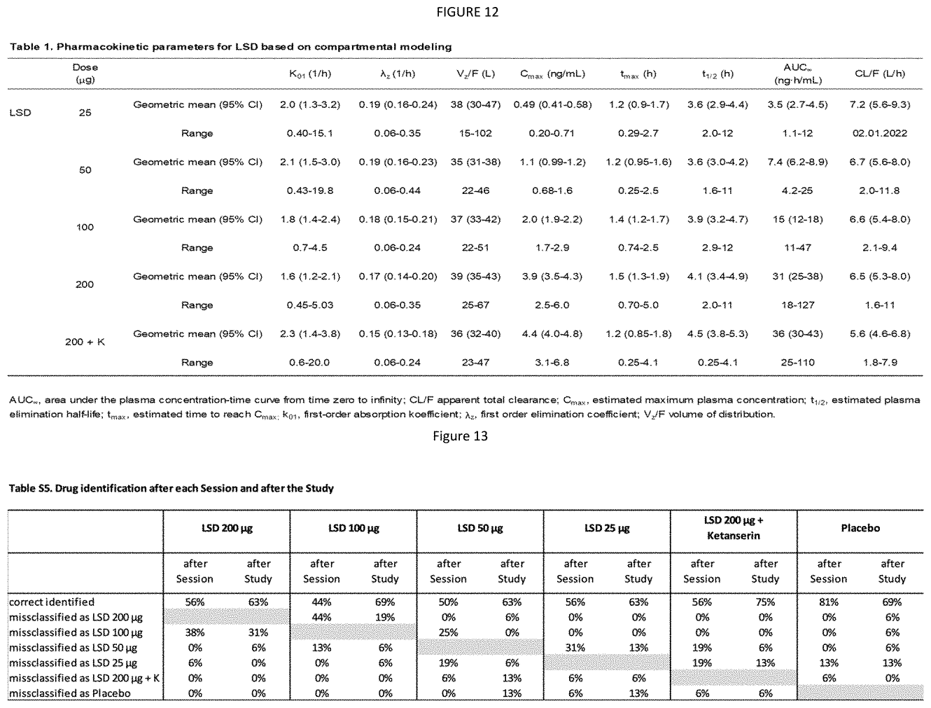

Blinding: Data on the participants' retrospective identification of the LSD dose condition are shown in FIG. 13. Ketanserin and LSD together were identified correctly or mistaken as a low dose of LSD but never mistaken for a high dose of LSD.

### Example 3

A clinical study administered ketanserin or placebo in a double-blind and randomized manner after a typical and fully psychoactive dose of LSD to document that the acute LSD effects can in practice be antagonized with ketanserin. Example data from three subjects taking part in the clinical study is presented here.

The key result is that ketanserin (40 mg) markedly shortened and also attenuated the acute subjective psychedelic response to a dose of LSD base of 100 μg (equivalent to 146 μg LSD tartrate 1:1). This finding confirms the practicability of using the present invention to block the effects of psychedelics.

Materials and Methods

Study design: The study used a double-blind, placebo-controlled, random-order 2-period cross-over design with 2 treatment conditions: 1) 100 μg LSD+ketanserin (40 mg) and 2) 100 μg LSD+placebo. Ketanserin or placebo was administered 1 hour after LSD. The washout periods between sessions were at least 10 days. The study was registered at ClinicalTrials.gov (NCT04558294).

Participants: Healthy subjects (men and women) were recruited. Participants who were younger than 25 years old were excluded from participating in the study. Additional exclusion criteria were age >65 years, pregnancy (urine pregnancy test at screening and before each test session), personal or family (first-degree relative) history of major psychiatric disorders (assessed by the Semi-structured Clinical Interview for Diagnostic and Statistical Manual of Mental Disorders, 4th edition, Axis I disorders by a trained psychiatrist), the use of medications that may interfere with the study medications (e.g. antidepressants, antipsychotics, sedatives), chronic or acute physical illness (abnormal physical exam, electrocardiogram, or hematological and chemical blood analyses), tobacco smoking (>10 cigarettes/day), lifetime prevalence of illicit drug use >10 times (except for Δ⁹-tetrahydrocannabinol), illicit drug use within the last 2 months, and illicit drug use during the study (determined by urine drug tests).

Study drugs: LSD (D-lysergic acid diethylamide base, high-performance liquid chromatography purity >99%; Lipomed AG, Arlesheim, Switzerland) was administered as an oral solution in units containing 100 μg LSD in 1 mL of 96% ethanol (Holze et al., 2019). Ketanserin was obtained as the marketed drug (KETENSIN® (Janssen)) and encapsulated with opaque capsules to ensure blinding. Ketanserin placebo consisted of identical opaque capsules filled with mannitol.

Study procedures: The study included a screening visit, two 13 hour test sessions (7:00 AM-8:00 PM), and an end-of-study visit. LSD was administered at 8:00 AM. Ketanserin (40 mg) or placebo was administered at 9:00 AM. The outcome measures were repeatedly assessed for 12 hours after LSD administration.

Subjective drug effects: Subjective effects were assessed repeatedly using visual analog scales (VASs) 0, 0.5, 1, 1.5, 2, 2.5, 3, 3.5, 4, 5, 6, 7, 8, 9, 10, 11, and 12 hours after LSD administration. The VASs were presented as 100-mm horizontal lines (0-100%), marked from “not at all” on the left to “extremely” on the right. The 5D-ASC scale (Dittrich, 1998; Studerus et al., 2010) was administered 12 hours after LSD administration to retrospectively rate alterations in waking consciousness induced by the drugs. Autonomic, adverse, and endocrine effects: Blood pressure, heart rate, and tympanic body temperature were repeatedly measured 1 hour before and 0, 0.5, 1, 1.5, 2.5, 3, 3.5, 4, 5, 6, 7, 8, 9, 10, 11, and 12 hours after LSD administration as previously described in detail (Hysek et al., 2010). Adverse effects were systematically assessed up to 12 hours after drug administration using the 66-item List of Complaints (Zerssen, 1976).

Results

Example data from three healthy subjects is shown as mean and SEM values to illustrate the effects of the present invention when put into practice in humans.

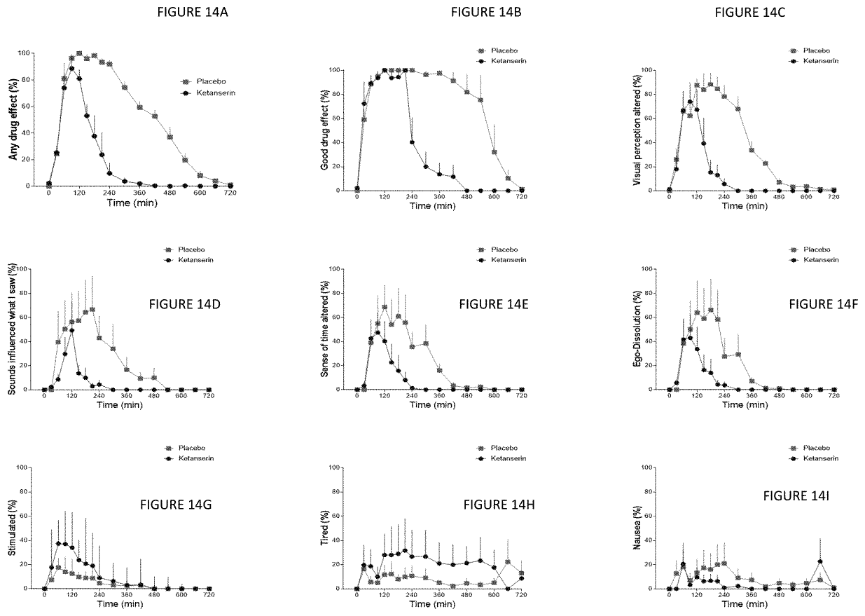

Subjective drug effects: Subjective effects over time on the VAS are shown in FIGS. 14A-141. Administration of ketanserin 1 hour after LSD markedly decreased the LSD effect compared with administration of placebo. The ketanserin effect on the subjective response to LSD set-in 1 hour after ketanserin administration and resulted in blockade of the LSD-typical effects within two hours to a nearly normal state. The duration of action of LSD was reduced by approximately 60% from 10 hours to 4 hours only with no recurrence of the LSD effect. Ketanserin very effectively reduced the typical mind altering effects of LSD including “any drug effect” (FIG. 14A), “good drug effect” (FIG. 14B), “visual perception alterations” (FIG. 14C), “auditory alterations” (not shown), synaesthetic effects (“sounds influenced what I saw” (FIG. 14D)), “alterations in the sense of time” (FIG. 14E), as well as the hallucinogen-typical experience of “ego-dissolution” (FIG. 14F). Ego-dissolution may sometimes lead to anxiety and anxiogenic effect can therefore also be expected to be reduced (anxiety was not present in any of the subjects tested in the present example). Ketanserin administered after LSD increased subjective feelings of stimulation compared with LSD alone (FIG. 14G) likely because these feelings are mediated via dopaminergic properties of LSD which were not antagonized by the selective serotonergic antagonist ketanserin. As expected, ketanserin added to LSD produced increased feelings of tiredness compared with the addition of placebo and representing a known side-effect of ketanserin (FIG. 14H). Ketanserin also tended to reduce nausea induced by LSD although more data is needed to validate this effect (FIG. 14I).

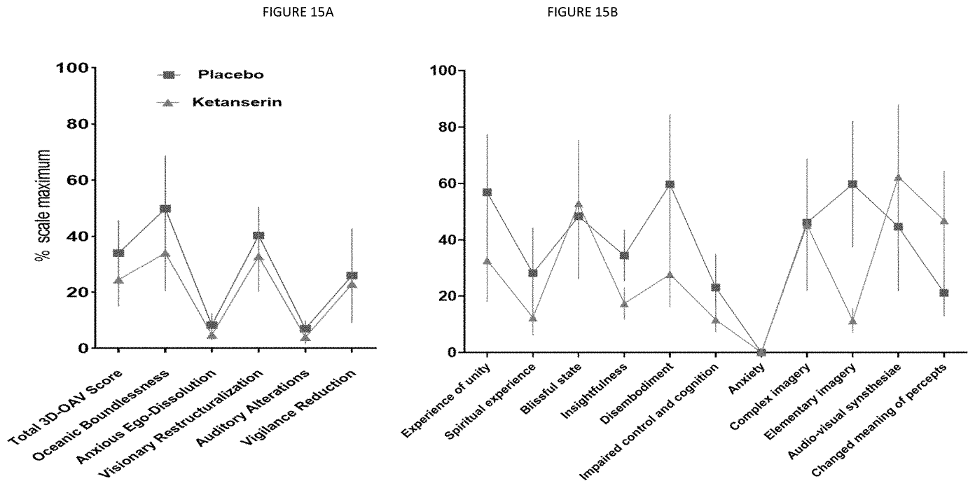

LSD-induced alterations of mind on two different sets of subscales are shown in FIGS. 15A and 15B. Administration of ketanserin after LSD reduced the subjective effects of LSD on the 5 Dimensions of Altered States of Consciousness (5D-ASC) Scale in comparison with placebo. Ketanserin reduced the total 3D-OAV score which reflects overall peak alterations of the mind on the three main scales produced by LSD by approximately 30%. The 3D-OAV score was 34% and 24% after placebo and ketanserin, respectively.

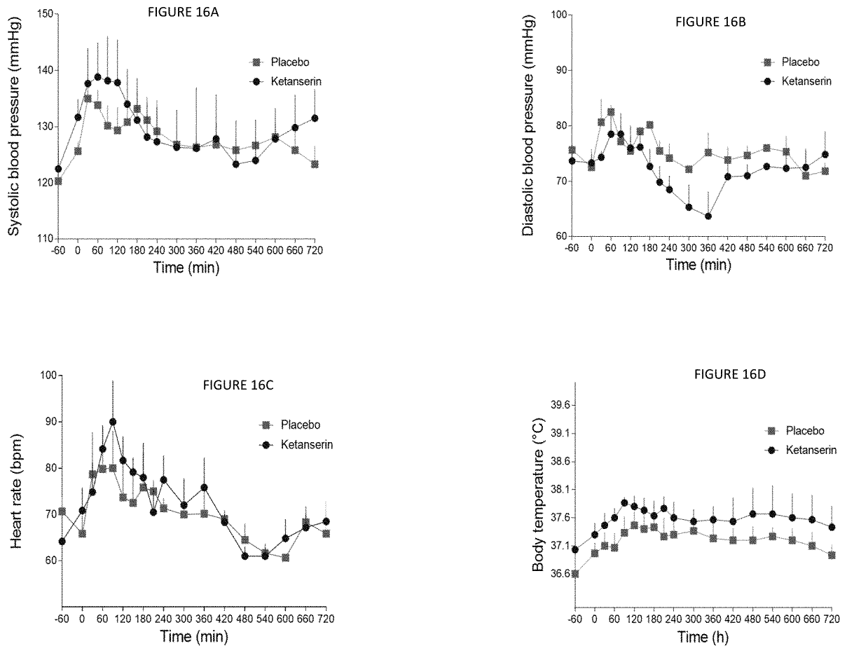

Cardiovascular effects of LSD over time are shown in FIGS. 16A-16D (systolic blood pressure (FIG. 16A), diastolic blood pressure (FIG. 16B), heart rate (FIG. 16C), and body temperature (FIG. 16D). Ketanserin had no relevant effects on the cardiovascular effects of LSD compared with adding placebo besides from a transient decrease in diastolic blood pressure needing further study.

When ketanserin was administered after LSD the total of reported complaints on the LC list up to 12 hours after LSD administration was (mean±SEM) 8.3±4 compared with 7±3.6 when placebo was administered after LSD. Thus, ketanserin did not reduce the total number of untoward effects reporting due to it having some adverse effects on its own.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Barrett F S, Johnson M W, & Griffiths R R (2015). Validation of the revised Mystical Experience Questionnaire in experimental sessions with psilocybin. J Psychopharmacol 29: 1182-1190.
2. Carhart-Harris R L, Bolstridge M, Day C M J, Rucker J, Watts R, Erritzoe D E, Kaelen M, Giribaldi B, Bloomfield M, Pilling S, Rickard J A, Forbes B, Feilding A, Taylor D, Curran H V, & Nutt D J (2017). Psilocybin with psychological support for treatment-resistant depression: six-month follow-up. Psychopharmacology.
3. Carhart-Harris R L, Bolstridge M, Rucker J, Day C M, Erritzoe D, Kaelen M, Bloomfield M, Rickard J A, Forbes B, Feilding A, Taylor D, Pilling S, Curran V H, & Nutt D J (2016). Psilocybin with psychological support for treatment-resistant depression: an open-label feasibility study. Lancet Psychiatry 3: 619-627.
4. Dittrich A (1998). The standardized psychometric assessment of altered states of consciousness (ASCs) in humans. Pharmacopsychiatry 31 (Suppl 2): 80-84.
5. Dolder P C, Schmid Y, Steuer A E, Kraemer T, Rentsch K M, Hammann F, & Liechti M E (2017). Pharmacokinetics and pharmacodynamics of lysergic acid diethylamide in healthy subjects. Clin Pharmacokinetics 56: 1219-1230.
6. Gasser P, Holstein D, Michel Y, Doblin R, Yazar-Klosinski B, Passie T, & Brenneisen R (2014). Safety and efficacy of lysergic acid diethylamide-assisted psychotherapy for anxiety associated with life-threatening diseases. J Nery Ment Dis 202: 513-520.
7. Gasser P, Kirchner K, & Passie T (2015). LSD-assisted psychotherapy for anxiety associated with a life-threatening disease: a qualitative study of acute and sustained subjective effects. J Psychopharmacol 29: 57-68.
8. Griffiths R R, Johnson M W, Carducci M A, Umbricht A, Richards W A, Richards B D, Cosimano M P, & Klinedinst M A (2016). Psilocybin produces substantial and sustained decreases in depression and anxiety in patients with life-threatening cancer: a randomized double-blind trial. J Psychopharmacol 30: 1181-1197.
9. Griffiths R R, Richards W A, McCann U, & Jesse R (2006). Psilocybin can occasion mystical-type experiences having substantial and sustained personal meaning and spiritual significance. Psychopharmacology 187: 268-283; discussion 284-292.
10. Grob C S, Danforth A L, Chopra G S, Hagerty M, McKay C R, Halberstadt A L, & Greer G R (2011). Pilot study of psilocybin treatment for anxiety in patients with advanced-stage cancer. Archives of general psychiatry 68: 71-78.
11. Hintzen A, & Passie T (2010) The pharmacology of LSD: a critical review. Oxford University Press: Oxford.
12. Holze F, Duthaler U, Vizeli P, Muller F, Borgwardt S, & Liechti M E (2019). Pharmacokinetics and subjective effects of a novel oral LSD formulation in healthy subjects. Br J Clin Pharmacol 85: 1474-1483.
13. Holze F, Vizeli P, Ley L, Müller F, Dolder P, Stocker M, Duthaler U, Varghese N, Eckert A, Borgwardt S, & Liechti M E (2020). Acute dose-dependent effects of lysergic acid diethylamide in a double-blind placebo-controlled study in healthy subjects. Neuropsychopharmacology doi: 10.1038/s41386-020-00883-6.
14. Hysek C M, Vollenweider F X, & Liechti M E (2010). Effects of a b-blocker on the cardiovascular response to MDMA (ecstasy). Emerg Med J 27: 586-589.
15. Janke W, & Debus G (1978) Die Eigenschaftsworterliste. Hogrefe: Gottingen.
16. Liechti M E (2017). Modern clinical research on LSD. Neuropsychopharmacology 42: 2114-2127.
17. Liechti M E, Dolder P C, & Schmid Y (2017). Alterations in conciousness and mystical-type experiences after acute LSD in humans. Psychopharmacology 234: 1499-1510.
18. Nichols D E (2016). Psychedelics. Pharmacological reviews 68: 264-355.
19. Passie T, Halpern J H, Stichtenoth D O, Emrich H M, & Hintzen A (2008). The pharmacology of lysergic acid diethylamide: a review. CNS Neurosci Ther 14: 295-314.
20. Preller K H, Herdener M, Pokorny T, Planzer A, Kraehenmann R, Stampfli P, Liechti M E, Seifritz E, & Vollenweider F X (2017). The fabric of meaning and subjective effects in LSD-induced states depend on serotonin 2A receptor activation Curr Biol 27: 451-457.
21. Rickli A, Moning O D, Hoener M C, & Liechti M E (2016). Receptor interaction profiles of novel psychoactive tryptamines compared with classic hallucinogens. European neuropsychopharmacology: the journal of the European College of Neuropsychopharmacology 26: 1327-1337.
22. Ross S, Bossis A, Guss J, Agin-Liebes G, Malone T, Cohen B, Mennenga S E, Belser A, Kalliontzi K, Babb J, Su Z, Corby P, & Schmidt B L (2016). Rapid and sustained symptom reduction following psilocybin treatment for anxiety and depression in patients with life-threatening cancer: a randomized controlled trial. J Psychopharmacol 30: 1165-1180.
23. Studerus E, Gamma A, & Vollenweider F X (2010). Psychometric evaluation of the altered states of consciousness rating scale (OAV). PLoS One 5: e12412.
24. Wacker D, Wang S, McCorvy J D, Betz R M, Venkatakrishnan A J, Levit A, Lansu K, Schools Z L, Che T, Nichols D E, Shoichet B K, Dror R O, & Roth B L (2017). Crystal structure of an LSD-bound human serotonin receptor. Cell 168: 377-389 e312.
25. Zerssen D V (1976) Die Beschwerden-Liste. Münchener Informations system. Psychis: München.

What is claimed is:

1. A composition for treating an individual while reducing acute effects, comprising effective amounts of a psychedelic drug and a duration shortening agent.

2. The composition of claim 1, wherein said psychedelic drug is a 5HT2A agonist chosen from the group consisting of LSD, psilocybin, mescaline, dimethyltryptamine (DMT), 2,5-dimethoxy-4-iodoamphetamine (DOI), 2,5-dimethoxy-4-bromoamphetamie (DOB), salts thereof, analogs thereof, and homologues thereof.

3. The composition of claim 1, wherein said psychedelic drug is present in an amount that provides an effect for at least 2 hours.

4. The composition of claim 3, wherein said psychedelic drug is present in an amount chosen from the group consisting of 0.01-1 mg LSD, 10-50 mg psilocybin, 100-800 mg mescaline, 20-100 mg DMT, 0.1-5 mg DOI, and 0.1-5 mg DOB.

5. The composition of claim 1, wherein said duration shortening agent is a 5HT2A receptor antagonist.

6. The composition of claim 5, wherein said duration shortening agent is chosen from the group consisting of ketanserin, salts thereof, analogs thereof, and homologs thereof.

7. The composition of claim 6, wherein said ketanserin is present in an amount of 5-100 mg.

8. The composition of claim 1, wherein said psychedelic drug and duration shortening agent are in dosage units chosen from the group consisting of separate dosage units, in the same dosage unit with the same release profiles, and in the same dosage unit with different release profiles.

9. A method of treating an individual with a psychedelic drug and reducing its acute duration of action, including the steps of:
administering a psychedelic drug to the individual;
administering a duration shortening and/or effect blocking agent to the individual; and
shortening and/or reducing the acute effects of the psychedelic drug.

10. The method of claim 9, wherein the duration shortening agent is administered 1 minute to 24 hours after administering the psychedelic drug.

11. The method of claim 9, wherein the psychedelic drug is a 5HT2A agonist chosen from the group consisting of LSD, psilocybin, mescaline, dimethyltryptamine (DMT), 2,5-dimethoxy-4-iodoamphetamine (DOI), 2,5-dimethoxy-4-bromoamphetamie (DOB), salts thereof, analogs thereof, and homologues thereof.

12. The method of claim 9, wherein the psychedelic drug is administered in an amount that provides an effect for at least 2 hours.

13. The method of claim 12, wherein the psychedelic drug is administered in an amount chosen from the group consisting of 0.01-1 mg LSD, 10-50 mg psilocybin, 100-800 mg mescaline, 20-100 mg DMT, 0.1-5 mg DOI, and 0.1-5 mg DOB.

14. The method of claim 9, wherein the duration shortening or/and effect blocking agent is a 5HT2A receptor antagonist.

15. The method of claim 14, wherein the duration shortening or/and effect blocking agent is chosen from the group consisting of ketanserin, salts thereof, analogs thereof, and homologs thereof.

16. The method of claim 15, wherein the ketanserin is administered in an amount of 5-100 mg.

17. The method of claim 9, wherein the psychedelic drug and duration shortening agent are in dosage units chosen from the group consisting of separate dosage units, in the same dosage unit with the same release profiles, and in the same dosage unit with different release profiles.

18. The method of claim 9, further including the step of reducing the time of subjective effects or/and reducing the amount of effects including any drug effect, bad drug effect, anxiety, ego-dissolution, and autonomic response measures by 10-100% compared with a treatment of the same amount of the psychedelic drug alone.

19. The method of claim 9, wherein said shortening step is accomplished by the duration shortening and/or effect reducing agent preventing interaction of the psychedelic drug with 5HT2A receptors.

20. The method of claim 9, wherein said shortening step is further defined as returning the individual to approximately a normal state.

21. The method of claim 9, further providing no recurrence of the psychedelic drug effects after the duration shortening agent is administered.

22. The method of claim 9, further including a step chosen from the group consisting of reducing time and/or degree of cognitive impairment due to the psychedelic drug, reducing time of treatment session supervision by medical personnel, reducing intensity and/or duration of anxiety or any other acute adverse effects in response to the psychedelic drug, reducing expected acute adverse effects intensity and/or duration due to inadvertent administration of a high dose of the psychedelic drug, reducing expected acute adverse effects intensity and/or duration due to intentional intake of the psychedelic drug, and reducing expected acute adverse effects duration and/or intensity due to intentional intake of the psychedelic drug in doses considered too high or producing too strong effects after administration.

23. A method of stopping the acute duration of action of a psychedelic drug in an individual, including the steps of:
administering a duration shortening and/or effect reducing agent to the individual after the individual has taken a psychedelic drug; and
stopping the acute effects of the psychedelic drug.

24. The method of claim 23, wherein the individual is experiencing an adverse effect due to the psychedelic drug.

25. The method of claim 23, wherein the individual has overdosed on the psychedelic drug.

26. The method of claim 23, wherein the duration shortening agent is administered 1 minute to 24 hours after administering the psychedelic drug.

27. The method of claim 23, wherein the psychedelic drug is a 5HT2A agonist chosen from the group consisting of LSD, psilocybin, mescaline, dimethyltryptamine (DMT), 2,5-dimethoxy-4-iodoamphetamine (DOI), 2,5-dimethoxy-4-bromoamphetamie (DOB), salts thereof, analogs thereof, and homologues thereof.

28. The method of claim 23, wherein the duration shortening and/or effect reducing agent is a 5HT2A receptor antagonist.

29. The method of claim 28, wherein the duration shortening and/or effect reducing agent is chosen from the group consisting of ketanserin, salts thereof, analogs thereof, and homologs thereof.

30. The method of claim 29, wherein the ketanserin is administered in an amount of 5-100 mg.

31. The method of claim 23, wherein said stopping step is accomplished by the duration shortening and/or effect reducing agent preventing interaction of the psychedelic drug with 5HT2A receptors.

32. The method of claim 23, wherein said stopping step is further defined as returning the individual to approximately a normal state.

33. The method of claim 23, further providing no recurrence of the psychedelic drug effects after the duration shortening agent is administered.
